(19) United States
(12) Patent Application Publication
(10) Pub. No.: US 2026/0241202 A1
(43) Pub. Date: Aug. 20, 2026

(54) RADIATION SPACER DEVICES, DELIVERY SYSTEMS, AND METHODS FOR THE PREVENTION OF COLLATERAL RADIATION

(71) Applicant: BARD PERIPHERAL VASCULAR, INC., Franklin Lakes, NJ (US)

(72) Inventors: Jordan ADDISON, Gilbert, AZ (US); Hiep DO, Chandler, AZ (US); Chad VAN LIERE, Phoenix, AZ (US)

(73) Assignee: BARD PERIPHERAL VASCULAR, INC., Franklin Lakes, NJ (US)

(21) Appl. No.: 19/099,313

(22) PCT Filed: Aug. 16, 2022
(86) PCT No.: PCT/US2022/074998

§ 371 Date: Jan. 28, 2025

(51) Int. Cl. A61N 5/10; A61B 17/00; A61M 25/10

(52) U.S. Cl. CPC A61N 5/1027; A61M 25/10182; A61M 25/10186; A61B 2017/00004; A61M 2025/1054; A61N 2005/1021

(57) ABSTRACT

A radiation spacer device includes an implantable balloon and a plurality of duckbill valves. The implantable balloon defines a cavity for holding a fluid therein. The implantable balloon has a flexible body and a neck. The neck defines an aperture into the cavity. The plurality of duckbill valves are disposed within the aperture and are serially nested within one another and arranged relative to one another to restrict movement of the fluid out of the cavity.

### BACKGROUND

### Field

The present disclosure generally relates to radiation spacer devices and delivery systems, and more specifically, to devices, systems, and methods for delivering a radiation spacer device.

### Technical Background

Fluid-filled balloon spacers that are used for implantation may be subject to leakage or other complications. For example, as pressure is exerted on the balloon, spacing between a target organ and an adjacent organ may fluctuate. In some circumstances, the balloon may deflate while a subject is still undergoing treatment. Each of these scenarios can render a spacer device less effective, resulting in less efficacy in protecting the adjacent organs or the need for follow-up procedures that can result in increased complications.

### SUMMARY

In one aspect, a radiation spacer device, includes an implantable balloon defining a cavity for holding a fluid therein, the implantable balloon having a flexible body and a neck, said neck defining an opening into the cavity, and a plurality of duckbill valves disposed within the opening into the cavity, where the plurality of duckbill valves are serially nested within one another and arranged relative to one another to restrict movement of the fluid out of the cavity.

The radiation spacer device may also include aspects where the implantable balloon is formed from a biodegradable polymer. The radiation spacer device may also include aspects where the flexible body is expandable in response to an increasing amount of fluid introduced to the cavity. The radiation spacer device may also include aspects where each duckbill valve of the plurality of duckbill valves includes a base portion defining a first lumen portion, and a bill portion extending distally from the base portion and defining a second lumen portion having a decreasing cross-sectional area from the first lumen portion to an outlet formed at a distal end of the duckbill valve, where the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity. The radiation spacer device may also include aspects where the bill portion of the second duckbill valve is angularly rotated relative to the bill portion of the first duckbill valve by 90 degrees. The radiation spacer device may also include aspects where the fluid includes a biodegradable hydrogel. The radiation spacer device may also include aspects where the radiation spacer device has a bioadhesive coating or physical mechanism for decreasing the mobility of the radiation spacer within an insertion site disposed on an exterior surface of the flexible body. Other technical features may be readily apparent to one skilled in the art from the following figures, descriptions, and claims.

In another aspect, a radiation spacer delivery system includes a radiation spacer device including an implantable balloon defining a cavity for holding a fluid therein, the implantable balloon having a flexible body and a neck, said neck defining an opening into the cavity, and a plurality of duckbill valves disposed within the opening into the cavity, where the plurality of duckbill valves are serially nested within one another and arranged relative to one another to restrict movement of the fluid out of the cavity. The radiation spacer delivery system also includes an injection assembly including an elongate member, and a syringe includes a chamber and a plunger, where the chamber is fluidically coupled to the elongate member. The radiation spacer delivery system also includes a fluid delivery apparatus including a housing, the housing including a cannula, a support member, and an actuator, and a detachment mechanism.

The radiation spacer delivery system may also include aspects where each duckbill valve of the plurality of duckbill valves includes a base portion defining a first lumen portion, and a bill portion extending distally from the base portion and defining a second lumen portion having a decreasing cross-sectional area from the first lumen portion to an outlet formed at a distal end of the duckbill valve, where the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity. The radiation spacer device may also include aspects where the bill portion of the second duckbill valve is angularly rotated relative to the bill portion of the first duckbill valve by 90 degrees. The radiation spacer delivery system may also include aspects where the detachment mechanism includes a resistive coil. The radiation spacer delivery system may also include aspects where the fluid is a biodegradable hydrogel. The radiation spacer delivery system may also include aspects where the implantable balloon is a biodegradable polymer. Other technical features may be readily apparent to one skilled in the art from the following figures, descriptions, and claims.

In one aspect, a method of protecting against collateral radiation of a non-targeted tissue, the method includes inserting a radiation spacer device between a targeted tissue, intended to receive radiation therapy and the non-targeted tissue, where the radiation spacer device includes an implantable balloon defining a cavity for holding a fluid therein, the implantable balloon having a flexible body and a neck, said neck defining an opening into the cavity, and a plurality of duckbill valves disposed within the opening into the cavity, where the plurality of duckbill valves are serially nested within one another and arranged relative to one another to restrict movement of the fluid out of the cavity. The method also includes expanding the implantable balloon to create a separation between the targeted tissue and the non-targeted tissue, thereby protecting the non-targeted tissue from the effect of the therapy applied to the targeted tissue.

The method may also include aspects where the targeted tissue is cancerous tissue and the non-targeted tissue is an adjacent organ. The method may also include aspects where the radiation spacer device is coupled to a fluid delivery apparatus. The method may also include aspects where the fluid delivery apparatus includes a housing and a detachment mechanism. The method may also include detaching the radiation spacer device from the fluid delivery apparatus using the detachment mechanism. The method may also include aspects where the detachment mechanism is a resistive coil. The method may also include aspects where detaching the radiation spacer device includes thermally ablating the neck of the implantable balloon with the resistive coil. The method may also include aspects where expanding the implantable balloon includes filling the cavity with the fluid. The method may also include aspects where the fluid is a biodegradable hydrogel. The method may also include aspects where filling the cavity with fluid includes passing the fluid from an injection assembly to the cavity. The method may also include aspects where the injection assembly includes an elongate member and a syringe, the syringe includes a chamber for holding the fluid and a plunger. The method may also include aspects where passing the fluid from the injection assembly to the cavity includes actuation of the plunger to dispense the fluid from the chamber, into the elongate member, and into the cavity. Other technical features may be readily apparent to one skilled in the art from the following figures, descriptions, and claims.

Additional features and advantages of the aspects described herein will be set forth in the detailed description which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a side view of an illustrative delivery system including an injection assembly, a radiation spacer device, and a fluid delivery apparatus according to one or more aspects shown and described herein;

FIG. 2 depicts a perspective view of the injection needle assembly of FIG. 1, including an elongate member and a syringe according to one or more aspects shown and described herein;

FIG. 3 depicts a perspective view of the elongate member of FIG. 2 according to one or more aspects shown and described herein;

FIG. 4 depicts a perspective view of the delivery system of FIG. 1 with the injection assembly decoupled from the fluid delivery apparatus and the radiation spacer device according to one or more aspects shown and described herein;

FIG. 5 depicts an exploded view of the fluid delivery apparatus and injection assembly of FIG. 1 according to one or more aspects shown and described herein;

FIG. 6 depicts a perspective view of the fluid delivery apparatus of FIG. 1 decoupled from the radiation spacer device of FIG. 1 according to one or more aspects shown and described herein;

FIG. 7 depicts an alternate perspective view of the delivery system of FIG. 1 according to one or more aspects shown and described herein;

FIG. 8 depicts a perspective view of the delivery system of FIG. 1 with an illustrative detachment mechanism according to one or more aspects shown and described herein;

FIG. 9 depicts a cross-sectional view of the radiation spacer device of FIG. 1 coupled to a fluid delivery apparatus and an illustrative detachment mechanism according to one or more aspects shown and described herein;

FIG. 10 depicts a perspective view of the radiation spacer device of FIG. 1 in an expanded state according to one or more aspects shown and described herein;

FIG. 11A depicts a perspective view of illustrative serially nested duckbill valves according to one or more aspects shown and described herein;

FIG. 11B depicts a cross-sectional view of the serially nested duckbill valves of FIG. 11A; and

FIG. 11C depicts a perspective view of the duckbill valves of FIG. 11A in a decoupled state according to one or more aspects shown and described herein.

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

The present disclosure, in one form, is related to radiation spacer devices that incorporate an implantable balloon with serially-nested duckbill valves for the separation of tissues to protect against collateral radiation as well as systems and methods that incorporate the same. The radiation spacer devices described herein include an implantable balloon that defines a cavity for holding a fluid. In addition, the radiation spacer devices described herein further include the implantable balloon having a flexible body and a neck, said neck defining an opening into the cavity, and a plurality of duckbill valves disposed within the opening into the cavity, where the plurality of duckbill valves are serially nested within one another and arranged relative to one another to restrict movement of the fluid out of the cavity.

Further a radiation spacer device described herein may be implanted for a length of a subject's treatment, but additional procedures increase the risk of complications. As such, the devices, systems, and methods described herein include biodegradable components that can be adapted to a subject's specific needs. As used herein “adapted” means that the radiation spacer device is particularly configured, shaped, and sized to meet the subject's specific anatomy and treatment goals. “Adapted” also means that the materials forming the implantable balloon and/or the fluid filling the balloon are selected for or tuned to a specific degradation profile that aligns with the subject's anticipated treatment length and/or needs.

The radiation spacer device disclosed herein is designed such that, in an expanded state, the device is capable of creating separation between a targeted tissue and a non-targeted tissue. As used herein, the term “separation” or “displacement” refers to filling a void between the targeted tissue and the non-targeted tissue or moving the targeted tissue and the non-targeted tissue such that the radiation spacer device creates and fills a void between the tissues. This space created by the radiation spacer device protects the non-targeted tissue from exposure or unintended side effects during treatment. The radiation spacer device can reduce harmful effects of radiation therapy, allow for improved targeting, allow for higher doses of radiation, and/or allow for shorter treatment times.

Further, the radiation spacer device disclosed herein is designed to maintain the expanded state for the length of intended treatment. To prevent deflation of the device, such as through loss of fluid, the radiation spacer device can include inflation media that gels to prevent loss of fluid, and closures that permit one directional flow into the radiation spacer device but prevent backflow.

Prostate cancer is the most common non-skin cancer diagnosed in men. Radiation therapy is an excellent treatment option for prostate cancer. However, radiation exposure can cause unintended side effects in adjacent organs. A fluid-filled balloon spacer can be implanted to avoid collateral radiation and minimize injury to nearby organs by providing a space between the target organ or tissue and nearby organs or tissues at risk.

As used herein, the term “targeted tissue” refers to a tissue or organ in need of radiation therapy or other treatment. As used herein, the term “non-targeted tissue” refers to a tissue or organ adjacent to the targeted tissue, where the non-targeted tissue is at risk of side effects from the treatment of the targeted tissue. In some embodiments, the non-targeted tissue is at risk of collateral radiation.

The device disclosed herein may also be used in other medical procedures and treatment, including, but not limited to, vessel occlusion, punctal occlusion, ductal occlusion, and other procedures and treatments that require obstructing a lumen in a subject. Additionally, the device may be used in medical procedures that require creating space in a subject, including but not limited to, orbital volume augmentation, dental procedures, tissue expansion for reconstructive surgery, vocal fold procedures, and the like.

An advantage of the present disclosure is that the biodegradable components reduce the need for follow up procedures. Additionally, the present disclosure provides devices that can be tailored to a subject's unique anatomy and treatment needs. Further, another advantage of the present disclosure is that radiation spacer devices are uniquely configured to prevent leakage, thereby allowing the space created between the targeted and non-targeted tissue to be maintained.

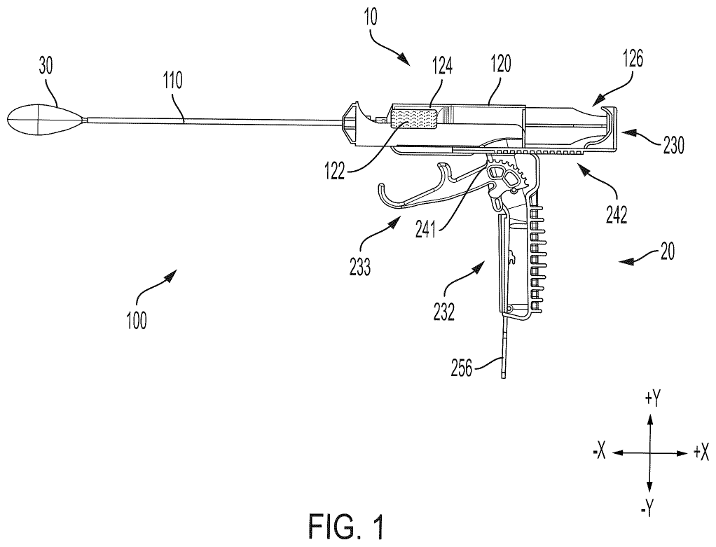

Turning now to the drawings, FIG. 1 depicts an illustrative delivery system 100 according to various aspects. The delivery system 100, in accordance with an aspect of the present invention, can be used in a radiation spacer device delivery procedure whereby a radiation spacer device 30 (e.g., a balloon) is delivered to a site intended to receive radiation therapy. The delivery system 100 has a proximal end that extends proximally (e.g., in the +x direction of the coordinate axes of FIG. 1) and a distal end that extends distally (e.g., in the −x direction of the coordinate axes of FIG. 1). The delivery system 100 generally includes an implantable radiation spacer device 30, an injection assembly 10, and/or a fluid delivery apparatus 20. A greater or fewer number of components may be included without departing from the scope of the present disclosure. The various components for the delivery system 100 are couplable together for the purposes of delivering a fluid to expand the radiation spacer device 30, as described herein.

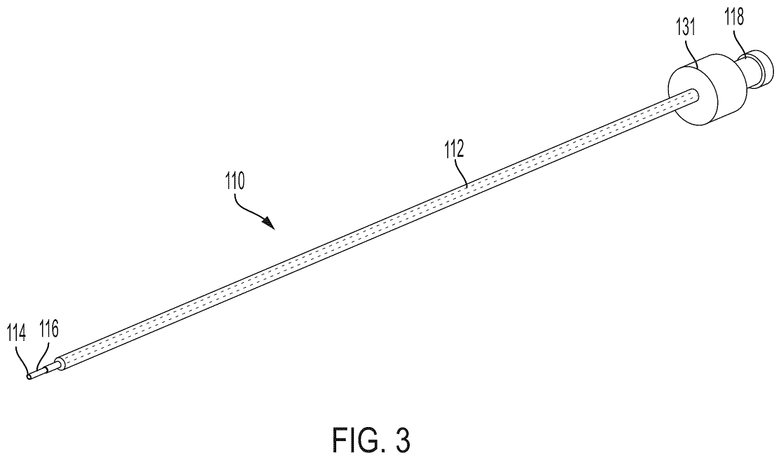

Referring jointly to FIGS. 1, 2, 3, and 4, the injection assembly 10 generally includes an elongate member 110 and a syringe 120. The injection assembly 10 has a distal end that extends distally (e.g., in the −x direction of the coordinate axes of FIG. 1) and a proximal end that extends proximally (e.g., in the +x direction of the coordinate axes of FIG. 1). In aspects, the elongate member 110 extends distally (e.g., in the −x direction of the coordinate axes of FIG. 1) from the syringe 120.

The elongate member 110 may generally be a hollow cylinder and may define an inflation lumen 112. The inflation lumen 112 may extend from an inflation port 118, disposed at the proximal end of the elongate member 110, through the length of the elongate member 110. The elongate member 110 is generally fluidly coupled to the radiation spacer device 30 to enable inflation, described in greater detail below.

In aspects, the inflation lumen 112 is configured to receive an inflation fluid 122, and to pass the inflation fluid 122 into the radiation spacer device 30 for expansion, as described in greater detail below. As used herein, the term “fluid”, refers to any flowable substance that has the ability to fill the radiation spacer device 30, such as, but not limited to, a biodegradable hydrogel, saline, contrast media, an injectable viscous fluid, and the like. In aspects, the inflation fluid 122 may further include contrasting agents, such as iodinated compounds, baritated compounds, fluorocarbons, echogenic compounds, anechoic compounds, gadolinium, radioactive isotopes, pain medication, pharmaceuticals, chemotherapeutics, and the like. In aspects, the inflation fluid 122 may further include biocompatible radiation shielding materials, including, but not limited to, polymer composite materials, tungsten, bismuth, antimony, and the like.

In aspects, the inflation fluid 122 is a biodegradable hydrogel. Any suitable hydrogel materials may be used. Illustrative examples of suitable hydrogel materials include, but are not limited to, albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. As used herein, “biodegradable” and/or “bioabsorbable” refers to a compound that can be absorbed by the surrounding or local tissue of a subject and/or degraded and absorbed by the tissue of the subject.

The hydrogel can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9, months, 10 months, 11 months, 12 months, 13 months, 14 months, 15 months, 16 months, 17 months, and 18 months. It should be understood that the time is merely a rough guide generally used to target appropriate formulation of the hydrogel.

The elongate member 110 may further define an outlet 114, positioned at a distal end of the elongate member 110 and coaxial with the inflation lumen 112, such that the outlet 114 is in fluid communication with the inflation lumen 112 and the radiation spacer device 30. In some aspects, the elongate member 110 may have a sharpened distal end 116.

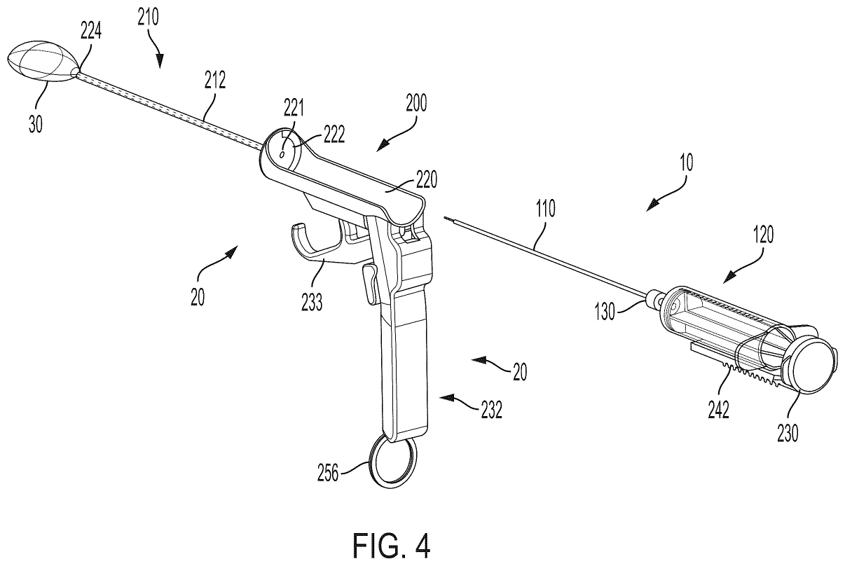

In some aspects, the elongate member 110 is sized to fit within a cannula lumen 212 of the fluid delivery apparatus 20, as depicted in FIG. 4 and discussed in greater detail below. As illustrated in FIGS. 1-4, the elongate member 110 may have a round cross-sectional shape, although it should be understood that the elongate member 110 may have any other suitable cross-sectional shape (e.g., rectangular). The elongate member 110 may be made of any suitable material. Non-limiting examples of suitable materials include, for example, polyurethanes, polyamides, polyimides, nylon, acetyl, polytetrafluoroethylene (PTFE), polypropylene, stainless steel, and the like, though any suitable material is contemplated and possible.

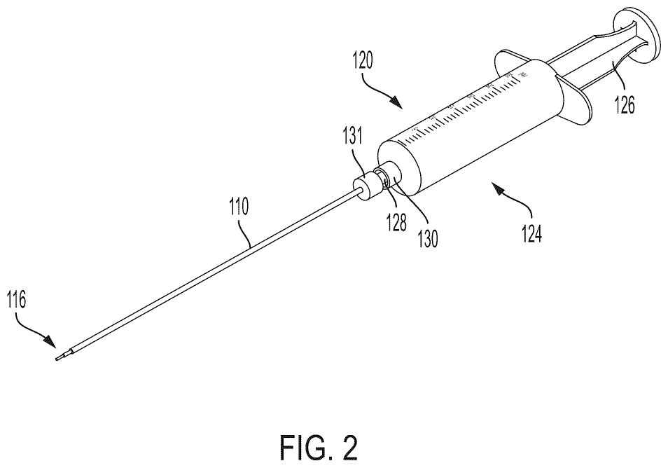

As illustrated in FIG. 2, the syringe 120 may be in fluid communication with the inflation lumen 112 of the elongate member 110. In aspects, the inflation port 118 is positioned at the proximal end of the elongate member 110. In aspects, the inflation port 118 fluidly connects the elongate member 110 to the syringe 120, as described in greater detail herein. As illustrated in FIGS. 1, 2, and 4, the syringe 120 may be coupled to the elongate member 110 and configured to dispense the inflation fluid 122. In aspects, the syringe 120 includes a chamber 124 and a plunger 126. The chamber 124 may be, for example, a cylindrical tube that is configured to hold the inflation fluid 122. The syringe 120 may also include an outlet port 128, disposed at the distal end of the chamber 124 and configured to dispense the inflation fluid 122. In some aspects, a distal end of the plunger 126 may be positioned within the chamber 124 and configured to dispense the inflation fluid 122 from the chamber 124 through the outlet port 128 when an operator depresses the plunger 126.

The elongate member 110 and the syringe 120 may include a connection mechanism 130. Corresponding connectors 131 may be disposed on the proximal end of the elongate member 110 and the distal end of the syringe 120. The corresponding connectors 131 may be generally shaped and sized to releasably interlock, forming the connection mechanism 130. The connection mechanism 130 may be used for coupling the syringe 120 to the elongate member 110. For example, in some aspects, the syringe 120 may include a quarter turn connector or other connector integrated with the distal end of the syringe 120. In some aspects, various components of the connection mechanism 130 are integrated with the chamber 124 such that the connection mechanism 130 and the chamber 124 are a single monolithic piece. However, it should be understood that this is merely illustrative and the various components of the connection mechanism 130 may be separate pieces that are permanently or semi-permanently joined with the syringe 120 and/or the elongate member 110 (e.g., permanently or semi-permanently joined with a distal coupling piece of the syringe).

The connector 131 disposed on the syringe 120 is generally located at the distal end of the chamber 124 such that various components of the connection mechanism 130 are positioned adjacent to the outlet port 128. In aspects, the elongate member 110 is configured to facilitate fluid communication with the output port 128 so as to receive the inflation fluid 122 from the chamber 124 and direct the inflation fluid 122 to the outlet 114 for delivery into the radiation spacer device 30, as described in greater detail below.

In aspects, when the corresponding connectors 131 are connected, the inflation port 118 of the elongate member 110 is aligned and sealed with the outlet port 128 of the syringe 120. Illustrative connection mechanisms include, but are not limited to, LUER-LOCK® (Bard Peripheral Vascular, Tempe AZ) connectors, LUER-SLIP® (Bard Peripheral Vascular, Tempe AZ) connectors, bayonet-style coupling connectors, L-beam coupling members, or the like. In aspects, a flow switch may be positioned between the chamber 124 and the elongate member 110 to allow for better control over the dispersal of the inflation fluid 122.

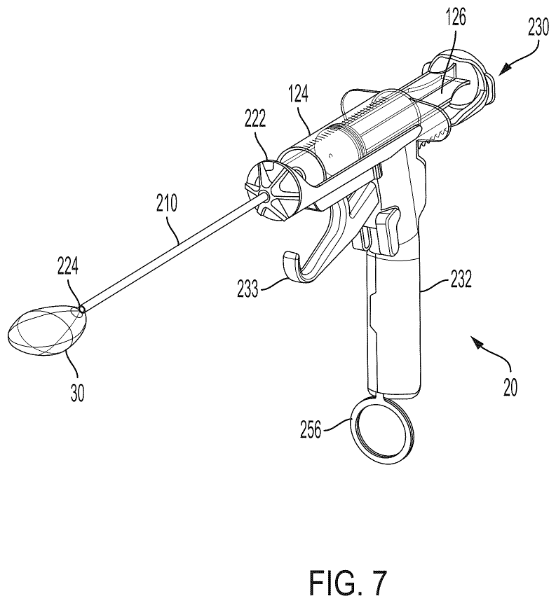

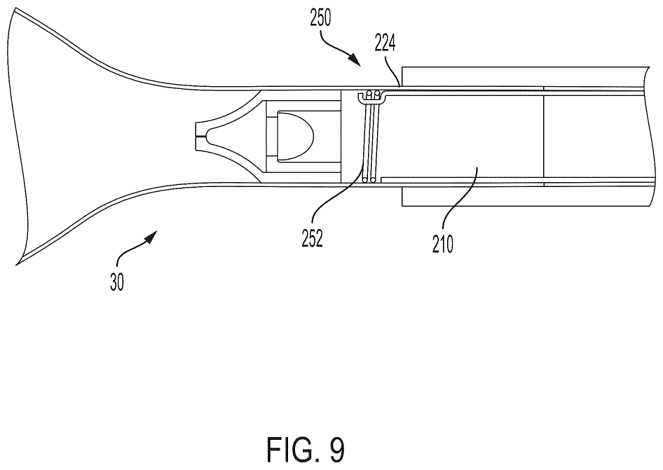

Referring now to FIGS. 1, 4, 5, and 7, the delivery system 100 generally includes a fluid delivery apparatus 20. The fluid delivery apparatus 20 generally includes a housing 200 coupled to the radiation spacer device 30 and/or the injection assembly 10. In aspects, such as shown in FIG. 9, the delivery system 100 may include a detachment mechanism 250. In aspects, the housing 200 includes a cannula 210, a support member 220, and/or an actuator 230.

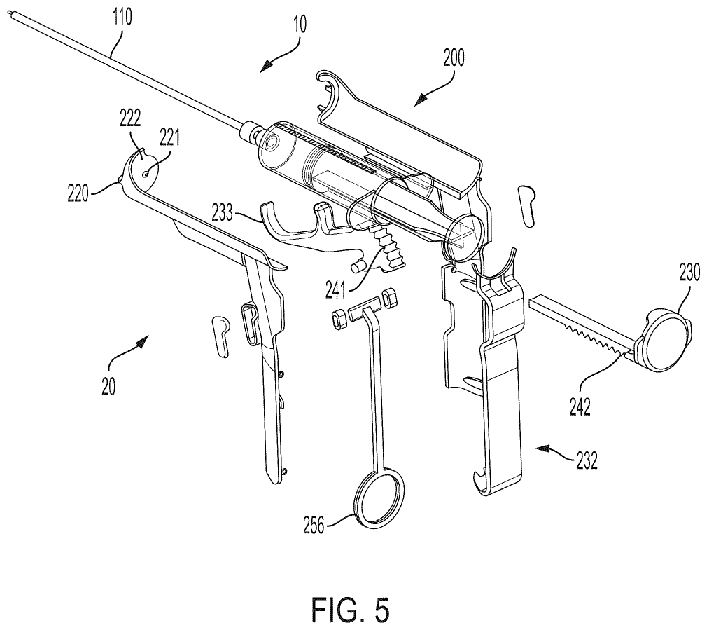

As illustrated in FIGS. 4 and 5, the housing 200 of the fluid delivery apparatus 20 may include a cannula 210. In aspects, the cannula 210 extends distally from the support member 220. In aspects, a distal end of the cannula 210 is coupled to the radiation spacer device 30 at an attachment point 224. In aspects, the cannula 210 defines a cannula lumen 212. The cannula lumen 212 may have an inner diameter which is larger than an outer diameter of the elongate member 110 such that the elongate member 110 may be inserted through the cannula lumen 212 and into the radiation spacer device 30. In aspects, the distal end of the elongate member 110 extends beyond the distal end of the cannula 210. In aspects, the attachment point 224 is disposed within the cannula lumen 212, such that a proximal end of the radiation spacer device 30 is attached to an interior wall of the cannula 210.

Still referring to FIGS. 4 and 5, the housing 200 may include a support member 220 configured to hold the syringe 120. In aspects, the support member 220 is configured to permit insertion of the elongate member 110. For example, in aspects, the support member 220 includes a distal plate 222, defining an opening 221. In aspects, the opening 221 is aligned with the cannula lumen 212 and is shaped such that the elongate member 110 extends through the opening 221 and into the cannula lumen 212.

The housing 200 may also generally include handle 232 and an actuator 230, configured to dispense the inflation fluid 122 from the chamber 124 of the syringe 120. As illustrated in FIGS. 1, 4, 5 and 7, the actuator 230 is coupled to the plunger 126 of the syringe 120 such that movement of the actuator 230 causes equivalent movement of the plunger 126. Example actuators include, but are not limited to, mechanical actuators, electro-mechanical actuators, pneumatic actuators, piezoelectric actuators, and hydraulic actuators.

In aspects, the actuator 230 may be configured for one-handed actuation by an operator, such as a clinician. In some aspects, as depicted in FIGS. 1,4, and 5, the actuator 230 may be coupled to a trigger 233. The trigger 233 may be disposed on the handle 232. In aspects, the trigger 233 is positioned for actuation while an operator is holding the handle 232. In aspects, such as depicted in FIG. 5, the trigger 233 includes gears 241 which are configured to move corresponding gears 242 of the actuator 230. The actuator 230 may be any suitable mechanism for moving the plunger 126, including, but not limited to tension actuator handles, and the like. In other aspects, the actuator 230 may be a handwheel or cam that can be actuated using an operator's thumb. Any type of appropriate actuators are contemplated and possible.

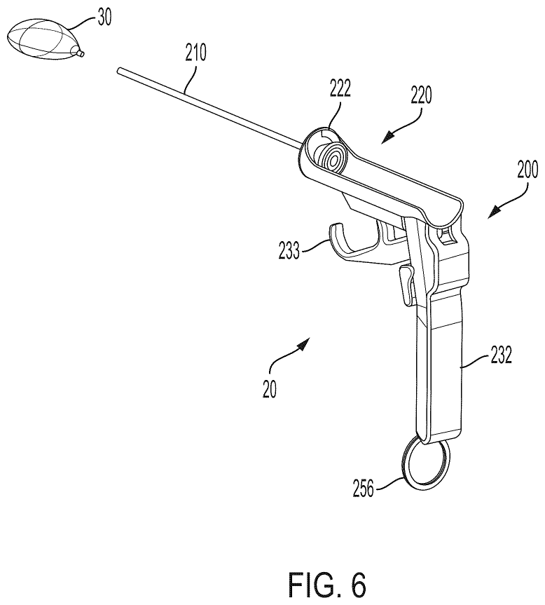

As illustrated in FIG. 6, in aspects, the fluid delivery apparatus 20 is separable from the injection assembly 10 and the radiation spacer device 30. In aspects, a decoupler 256 disengages the actuator 230 from the trigger 233. After inflation of the radiation space device 30, the injection assembly 10 can be removed from the housing 200 with the actuator 230, such as depicted in FIG. 4. After the radiation spacer device 30 has been filled with the inflation fluid 122, as discussed in further detail below, the radiation spacer device 30 can be decoupled from the fluid delivery apparatus 20, thereby allowing the radiation spacer device 30 to remain in place during radiation therapy, while the fluid delivery apparatus 20 and/or injection assembly 10 are removed from the body. In aspects, the fluid delivery apparatus 20 is separable from the radiation spacer device 30 using a detachment mechanism 250. Any type of detachment mechanism known in the art is contemplated and possible. Non-limiting examples of methods and devices to separate the radiation spacer device 30 from the fluid delivery apparatus 20 include mechanical, electrical, thermal, chemical, hydraulic, or sonic mechanisms.

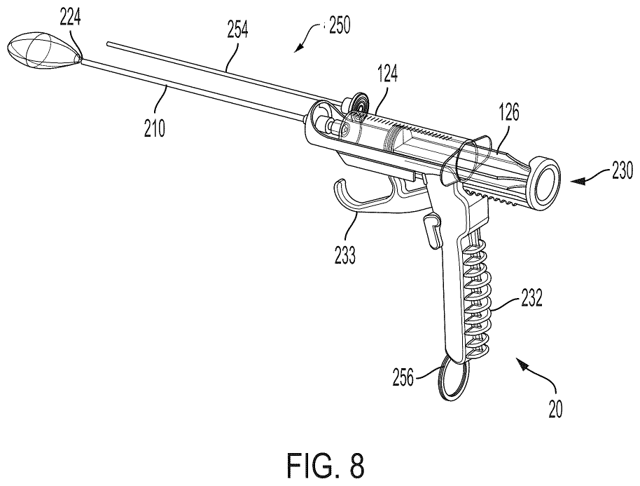

In some aspects, such as depicted in FIGS. 8 and 9, the radiation spacer device 30 can be detached from the cannula 210 using mechanical means. In aspects, radiation spacer device 30 can be separated from the fluid delivery apparatus 20 by a number of mechanical methods that cut, tear, or otherwise physically degrade a portion of the radiation spacer device 30 to separate the radiation spacer device 30 from the fluid delivery apparatus 20. This may be accomplished by exerting a force against the attachment point 224. In aspects, the detachment mechanism 250 may include a push rod 254. In aspects, the push rod 254 has a diameter smaller than the diameter of the cannula lumen 212. After removal of the injection assembly 10, the push rod 254 can be inserted through the cannula lumen 212. The diameter of the push rod 254 may be sufficient to interact with the radiation spacer device 30, and exert a force sufficient to cause the radiation spacer device 30 to detach from the attachment point 224. This allows an operator to remove the radiation spacer device 30 after inflation and allow it to remain in a subject.

As illustrated in FIG. 9, the detachment mechanism 250 can include a resistive coil 252, communicatively coupled with an electrical conductor (not shown), to decouple the radiation spacer device 30 using thermal ablation. In aspects, the detachment mechanism 250 may include a release mechanism causing the radiation spacer device 30 to detach from the cannula 210. In embodiments, the release mechanism may require actuation from the operator. Any appropriate release mechanism is contemplated and possible, including, but not limited to buttons, toggles, switches, triggers, handles, levers, pedals, and the like.

In aspects, the detachment mechanism 250 is communicatively coupled to the electrical conductor, such that engaging the release mechanism initiates the electrical current. As used herein, the term “communicatively coupled” means that coupled components are capable of exchanging data signals with one another such as, for example, electrical signals via conductive medium, electromagnetic signals via air, optical signals via optical waveguides, and the like.

The resistive coil 252 may be formed using any conductive material, including but not limited to, nickel chromium (nichrome), copper, stainless steel, titanium, zirconium, nickel titanium (Nitinol), ALUMEL® (Concept Alloys, Inc., Whitmore Lake, Michigan) iron-chromium-aluminum alloys, such as KANTHAL® (Sandvik Intellectual Property AB, Stockholm, Sweden), CHROMEL® (Concept Alloys, Inc., Whitmore Lake, Michigan), iron-nickel alloys, nickel-cobalt ferrous alloys, such as KOVAR® (CRS Holdings, Inc. Delaware), combinations or alloys of the same and the like, though any conductive material is contemplated and possible. After expansion of the radiation spacer device 30, a clinician can engage the release mechanism, triggering the electrical conductor to generate an electrical current, which is passed through the resistive coil 252, resulting in heating and detachment of the radiation spacer device 30.

Referring now to FIGS. 1 and 9, the delivery system 100 generally includes a radiation spacer device 30. The radiation spacer device 30 may include an implantable balloon 300. The implantable balloon 300 may include a flexible body 302, configured to expand or inflate such that in an expanded state, the flexible body 302 defines a cavity 304 configured to hold the inflation fluid 122. In aspects, the implantable balloon 300 may be in fluidic communication with the inflation lumen 112 via a neck 306 of the implantable balloon 300.

In aspects, the radiation space device 30 is coupled to the distal end of the cannula 210. The implantable balloon 300 may be attached to, or engaged with, the fluid delivery apparatus 20 in a variety of ways. For example, the implantable balloon 300 may be affixed to the cannula 210 by friction, using an adhesive, welding, soldering, clamping, or any other attachment method known in the art. As used herein the term, “balloon” refers to any expandable device having an inflated or expanded state and a deflated state such that in an inflated state, the device has an interior volume and in a deflated state, the device has substantially no interior volume. The terms deflated, collapsed, and forms thereof may be used interchangeably to refer to the implantable balloon 300 prior to filling the implantable balloon 300 with the inflation fluid 122. The terms expand, inflate, and forms thereof may be used interchangeably to refer to the action of changing the implantable balloon 300 from the deflated state to the expanded state.

The implantable balloon 300 may be formed from any suitable means, including but not limited to thermo-processing, extrusion blow molding, injection blow molding, solution dip coating on a pre-molded lost wax parison, and/or lamination of film on pre-molded lost wax parison, electrospinning, and the like. The shape and dimensions of the implantable balloon 300 may be adaptable to fit a subject's particular anatomy or treatment needs. In aspects, the implantable balloon 300 can be fabricated in any shape suitable for tissue displacement. Examples of suitable shapes include, but are not limited to, cylindrical, spherical, oblong, pear, fusiform, discoid, and triangular, though other shapes are contemplated and possible.

The implantable balloon 300 may formed from any biocompatible material. In aspects, the biocompatible material may be a biodegradable polymer. As set forth herein, a biodegradable polymer may include a polymer that is well-tolerated and/or non-reactive when contacted to a subject or immune-reactive cells thereof and is prone to erosion and/or enzymatic degradation and/or dissolution within the subject or the circulatory system thereof over a course of time. Biodegradable polymers allow for the elimination of the radiation spacer device 30 after the treatment has concluded, eliminating the need for a removal procedure. Illustrative examples of biodegradable polymers include, but are not limited to, polylactic acid polymers (PLA, PLLA, PDLA, PDLLA), polycaprolactone (PCL), poly lactic-co-glycolic Acid (PLGA), poly(ethylene glycol) methyl ether-block-poly(lactide-co-glycolide) (PLGA-b-mPEG), poly-4-hydroxybutyrate (P4HB) combinations thereof, and the like.

In aspects, the materials forming the implantable balloon 300 may be adapted based on the desired mechanical properties (e.g. ductility, malleability, plasticity, etc.) of the radiation spacer device 30 in vivo, as well as the intended length of treatment. For example, a biodegradable polymer can be selected that begins to degrade at the conclusion of the radiation therapy. Degradation rate and thus polymer selection may be determined according to the use of the radiation spacer device 30. Molecular weight of the polymers, crystalline structure, and other various properties can all be considered when determining appropriate polymers for formation of the implantable balloon 300.

In aspects when the inflation fluid 122 is a biodegradable hydrogel, both the implantable balloon 300 and the inflation fluid 122 can be formulated to begin degrading at the end of anticipated radiation therapy. In aspects, this is up to 18 months, for example from the range of about 0 months to about 18 months, including about 1 month, 2, months, 3 months, 4, months, 5 months, 6 months, 7 months, 8 months, 9, months, 10 months, 11 months, 12 months, 13 months, 14 months, 15 months, 16 months, 17 months, and 18 months. It should be understood that the time is merely a rough guide generally used to target appropriate formulation of the implantable balloon 300.

In addition, the material forming the implantable balloon 300 should be flexible enough to enable expansion of the flexible body 302. In aspects, additives may be included in the material forming the implantable balloon 300 to enhance desired properties. For example, plasticizers, such as triethyl citrate, glyceryl triacetate, acetyl triethyl citrate, polyethylene glycol 400, diethyl phthalate, tributyl citrate, acetylated monoglycerides, glycerol, fatty acid esters, propylene glycol, dibutyl phthalate, combinations thereof, and the like, may be added to increase flexibility.

In aspects, the implantable balloon 300 may include a bioadhesive coating or other physical mechanism (e.g. hooks, bumps, ridges, etc.) disposed on an exterior surface of the flexible body 302 which can decrease its mobility within the insertion site. This feature is important to minimize movement of the radiation spacer device 30 from the implantation site thereby ensuring protection for the non-treated tissue. As used herein, a bioadhesive may refer to a natural or synthetic material that can adhere to a biological surface, such as tissue.

In aspects, a bioadhesive may form a continuous or discontinuous film between the radiation spacer device 30 and a biological surface. By way of example, a bioadhesive may include hydroxypropyl methylcellulose, ethyl cellulose, hydroxypropyl cellulose, polyvinyl pyrimidine, polyvinyl alcohol, chitosan, polymethacrylate copolymers, silicones, polydimethylsiloxanes, acrylate copolymers, octylacrylamide copolymer, octisalate, combinations thereof, and the like.

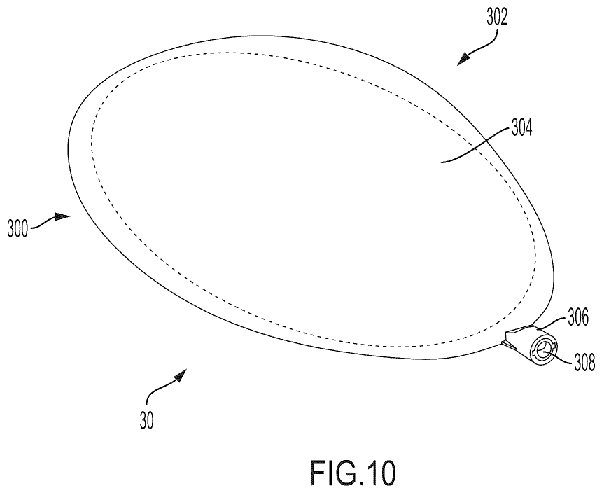

As illustrated in FIG. 10, the implantable balloon 300 generally includes a neck 306 defining an aperture 308 into the cavity 304. In aspects, the neck 306 may operably couple the distal end of the fluid delivery apparatus 20 to the radiation spacer device 30. As discussed above, a variety of attachment methods known in the art are contemplated and possible. In aspects, the neck 306 may define an aperture 308 for the passage of the inflation fluid 122 into the cavity 304 of the implantable balloon 300. The distal end of the elongate member 110 extends through the cannula lumen 212 and into the aperture 308, thereby fluidly coupling the inflation lumen 112 to the cavity 304.

In aspects, the aperture 308 may be in fluid communication with the elongate member 110 and the syringe 120 such that the inflation fluid 122 may be dispensed from the chamber 124 by actuation of the plunger 126 and delivered to the cavity 304 through the inflation lumen 112.

In aspects, the cavity 304 can be filled with the inflation fluid 122 to expand or inflate the flexible body 302. In aspects, the flexible body 302 is expandable in response to an increasing amount of inflation fluid 122 introduced to the cavity 304. Expansion of the flexible body 302 occurs after the radiation spacer device 30 is inserted into a treatment location, such as between the targeted tissue and non-targeted tissue. While in the deflated state, the radiation spacer device 30 may be inserted through any appropriate insertion method (e.g. an introducer sheath). A clinician may use any appropriate guide, including but not limited to ultrasound guidance, guidewires, and the like to facilitate insertion and placement of the radiation spacer device 30.

Once positioned, expansion of the flexible body 302 may be conducted to create separation between the adjacent tissues. In aspects, the flexible body 302 is expanded by the movement of inflation fluid 122 from the elongate member 110 to the cavity 304 via the aperture 308 in the neck 306 of the implantable balloon 300. The inflation fluid 122 enables the radiation spacer device 30 to conform to the displaced tissue and create better separation between the targeted and non-targeted tissues in the expanded configuration. In addition to the separation, the inflation fluid 122 may also act as a barrier against heat and/or radiation, serving to further protect the non-targeted tissue.

After the implantable balloon 300 is expanded, the radiation spacer device 30 is detached from the fluid delivery apparatus 20. In aspects, after expansion of the implantable balloon 300, the distal end of the cannula 210 is withdrawn from the neck 306 of the implantable balloon 300 to separate fluid delivery apparatus 20 from the radiation spacer device 30, allowing the fluid delivery apparatus 20 to be removed while leaving the radiation spacer device 30 in place, in its expanded state.

To maintain appropriate space between the targeted tissue and the non-targeted tissue, the radiation spacer device 30 needs to maintain volume for the anticipated length of treatment. In aspects, the expanded state is maintained by closing the aperture 308, thereby preventing deflation of the implantable balloon 300. In aspects, the inflation fluid 122 also aids in preventing deflation, such as by using the hydrogels as discussed above.

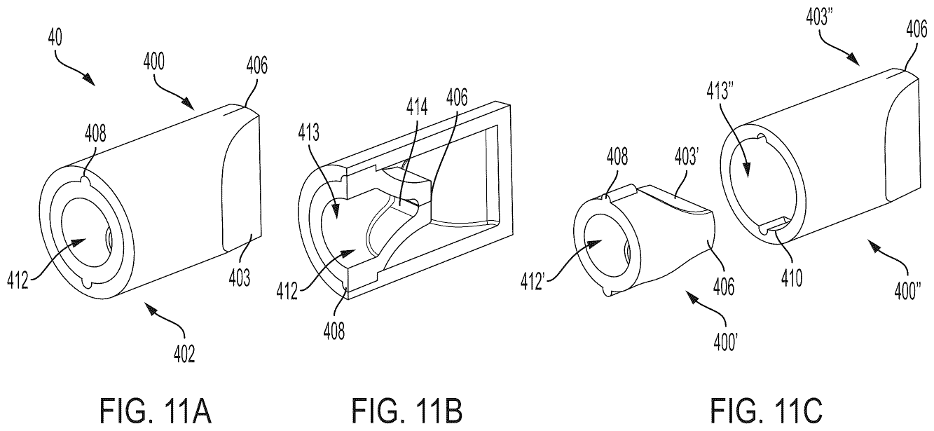

In some aspects, closing of the aperture 308 can be accomplished through a valve assembly 40, such as depicted in FIGS. 10 and 11A-C. As used herein, “check valve” and “duckbill valve” refer to valves that permit single directional flow of a fluid while inhibiting reverse flow and are used interchangeably. In aspects, such as depicted in FIGS. 11A-11C, one or more duckbill valves 400 may be disposed in the neck 306 of the implantable balloon 300. In aspects, the radiation spacer device 30 may have a plurality of duckbill valves 400 disposed in the neck 306 of the implantable balloon 300. In aspects, the neck 306 of the implantable balloon 300 may be reinforced with a tube sheath (not pictured) of biodegradable material to provide protection and support for the valve assembly 40.

In aspects, each duckbill valve 400 has a distal end that extends distally (e.g., in the −x direction of the coordinate axes of FIG. 1) and a proximal end that extends proximally (e.g., in the +x direction of the coordinate axes of FIG. 1. Each duckbill valve 400 may have a base portion 402 and a bill portion 403. In aspects, the base portion 402 may have a generally circular cross-sectional shape, though other shapes are contemplated and possible. In aspects, the bill portion 403 may have a generally flattened shape, as compared to the base portion 402. In aspects, the bill portion 403 extends distally from the base portion 402.

The duckbill valve 400 may define a lumen 412. In aspects, the lumen 412 is in fluidic communication with the cavity 304 and the inflation lumen 112. In aspects the distal end of the elongate member 110 is disposed within the lumen 412 to enable the inflation fluid 122 to expand the implantable balloon 300. In aspects, the base portion 402 defines a first lumen portion 413 and the bill portion 403 defines a second lumen portion 414. In aspects, the second lumen portion 414 may have a decreasing cross-sectional area from the first lumen portion 413.

The duckbill valves 400 can be configured to prevent fluid leakage from the implantable balloon 300. In aspects, the bill portion 403 defines an outlet 406 at the distal end of the duckbill valve 400. The outlet 406 may be any acceptable shape (e.g. a slit) that allows an open configuration and a closed configuration of the bill portion 403. As the inflation fluid 122 passes through the duckbill valve 400, the inflation fluid 122 creates pressure on the bill portion 403, holding the bill portion 403 in the open configuration. When the flow of the inflation fluid 122 is stopped, the base portion 403 returns to the flattened shape, e.g. the closed configuration. The closed configuration prevents the inflation fluid 122 from leaking out of the cavity 304. In aspects, the duckbill valves 400 are arranged such that the radiation spacer device 30 does not leak when fully compressed. Such testing, including tension and compression testing can be performed using any suitable method known in the art, including but not limited to Instron testing, and the like.

The duckbill valves 400 can be formed using a variety of conventional molding techniques known in the art (e.g. injection molding). In aspects, the duckbill valves 400 can be formed of biodegradable polymers. In aspects, the duckbill valves 400 may be made from the same materials as the implantable balloon 300. The duckbill valves 400 may be made of any materials that have a similar degradation profile to the implantable balloon 300. Any suitable materials are contemplated and possible.

In aspects, the plurality of duckbill valves 400 can provide a secure interlocking between the valves, such as by serially nesting, as depicted in FIGS. 11A and 11B. Although two serially nested duckbill valves 400 are depicted, it is noted that any additional number of valves is contemplated by the present disclosure. In aspects, the bill portion 403′ of a first duckbill valve 400′ extends into and is retained in the first lumen portion 413″ of a second duckbill valve 400″. When inflation fluid 122 is dispensed from the injection assembly 10, the inflation fluid 122 will force both bill portions 403′ and 403″ into the open configuration.

In aspects, the bill portion 403′ of the first duckbill valve 400′ is angularly rotated relative to the bill portion 403″ of the second duckbill valve 400″. The arrangement further restricts movement of the inflation fluid 122 out of the cavity 304. In aspects, the bill portion 403′ of the first duckbill valve 400′ is angularly rotated relative to the bill portion 403″ of the second duckbill valve 400″ by any appropriate angle, such as from about 15 degrees to 90 degrees, including 15 degrees, 30 degrees, 45 degrees, 60 degrees, 75 degrees, and 90 degrees. In aspects, the angular rotation allows the inflation fluid 122 to force both bill portions 403′ and 403″ into the open configuration. Once the flow of inflation fluid 122 stops, the bill portions 403′ and 403″ return to the closed configuration.

The plurality of duckbill valves 400 may be retained in the nested position using any suitable means, including, but not limited to, adhesives, welding, and the like. In embodiments, the plurality of duckbill valves 400 are formed to create a secure, interlocking connection between the duckbill valves 400. As illustrated in FIGS. 11A-11C, the plurality of duckbill valves 400 can be formed with a series of tongues 408 and grooves 410 which hold the plurality of duckbill valves 400 in the nested configuration.

In aspects, the valve assembly 40 remains in the radiation spacer device 30 after detachment from the fluid delivery apparatus 20. In the absence of a stiffer tubular support, slight distortions or force against the implantable balloon 300 may open a duckbill valve 400 and allow leakage of the inflation fluid 122. However, in aspects the angularly rotated arrangement may allow the pressure vector to open the second duckbill valve 400″ but close the first duckbill valve 400′. For example, even if the second duckbill valve 400″ were to fail to return to the closed configuration, thereby allowing reverse flow of the inflation fluid 122, the pressure created by the back flow would reinforce the closed configuration of the first bill portion 403′. Further, moving the radial force vector around the circumference of the duckbill valves 400 allows proportionally constant sealing action where the plurality of duckbill valves 400 exchange the dominance of sealing.

The following embodiments also relate to the present disclosure:

In a first embodiment, the present disclosure relates to a radiation spacer device comprising an implantable balloon defining a cavity for holding a fluid therein, the implantable balloon having a flexible body and a neck, said neck defining an aperture into the cavity; and a plurality of duckbill valves disposed within the aperture, wherein the plurality of duckbill valves are serially nested within one another and arranged relative to one another to restrict movement of the fluid out of the cavity.

In a second embodiment, the present disclosure relates to the radiation spacer device of the previous embodiment wherein the implantable balloon is formed from a biodegradable polymer.

In a third embodiment, the present disclosure relates to the radiation spacer device of any of the previous embodiments wherein the implantable balloon is expandable in response to an increasing amount of fluid introduced to the cavity.

In a fourth embodiment, the present disclosure relates to the radiation spacer device of any of the previous embodiments wherein each duckbill valve of the plurality of duckbill valves comprises a base portion defining a first lumen portion; and a bill portion extending distally from the base portion and defining a second lumen portion having a decreasing cross-sectional area from the first lumen portion to an outlet formed at a distal end of the duckbill valve; wherein the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity.

In a fifth embodiment, the present disclosure relates to the radiation spacer device of any of the previous embodiments wherein the bill portion of the second duckbill valve is angularly rotated relative to the bill portion of the first duckbill valve by 90 degrees.

In a sixth embodiment, the present disclosure relates to the radiation spacer device of any of the previous embodiments wherein the fluid comprises a biodegradable hydrogel. The fluid may also include contrast agents, biocompatible radiation shielding materials, pharmaceuticals, chemotherapeutics and/or combinations thereof.

In a seventh embodiment, the present disclosure relates to the radiation spacer device of any of the previous embodiments, wherein the radiation spacer device has a bioadhesive coating or physical mechanism for decreasing the mobility of the radiation spacer within an insertion site disposed on an exterior surface of the flexible body.

In an eighth embodiment, the present disclosure relates to a radiation spacer delivery system, comprising: a radiation spacer device comprising: an implantable balloon defining a cavity for holding a fluid therein, the implantable balloon having a flexible body and a neck, said neck defining an opening into the cavity, and a plurality of duckbill valves disposed within the opening into the cavity, wherein the plurality of duckbill valves are serially nested within one another and arranged relative to one another to restrict movement of the fluid out of the cavity; an injection assembly comprising: an elongate member, and a syringe comprising a chamber and a plunger, wherein the chamber is fluidically coupled to the elongate member; and a fluid delivery apparatus comprising: a housing, comprising a cannula, a support member, and an actuator, and a detachment mechanism.

In a ninth embodiment, the present disclosure relates to the radiation spacer delivery system of the previous embodiment, wherein the radiation spacer device is any of the first through the seventh embodiments.

In a tenth embodiment, the present disclosure relates to the radiation delivery system of any of the previous embodiments, wherein the radiation spacer device is positioned at the distal end of the radiation delivery system. The radiation spacer device is optionally attached to the fluid delivery apparatus, such as inside the cannula.

In an eleventh embodiment, the present disclosure relates to the radiation delivery system of any of the previous embodiments, wherein the elongate member is positioned proximal to the radiation spacer device.

In a twelfth embodiment, the present disclosure relates to the radiation delivery system of any of the previous embodiments wherein the elongate member is positioned distal to the syringe.

In a thirteenth embodiment, the present disclosure relates to the radiation delivery system of any of the previous embodiments wherein the actuator is coupled to a trigger via corresponding gears, such that depression of the trigger by an operator causes the actuator to depress the plunger.

In a fourteenth embodiment, the present disclosure relates to the radiation delivery system of any of the previous embodiments wherein each duckbill valve of the plurality of duckbill valves comprises: a base portion defining a first lumen portion; and a bill portion extending distally from the base portion and defining a second lumen portion having a decreasing cross-sectional area from the first lumen portion to an outlet formed at a distal end of the duckbill valve; wherein the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity.

In a fifteenth embodiment, the present disclosure relates to the radiation delivery system of any of the previous embodiments, wherein the bill portion of the second duckbill valve is angularly rotated relative to the bill portion of the first duckbill valve by 90 degrees.

In a sixteenth embodiment, the present disclosure relates to the radiation delivery system of any of the previous embodiments, wherein the detachment mechanism comprises a resistive coil. The resistive coil may be optionally disposed within the cannula of the fluid delivery apparatus. Further, the resistive coil may be disposed at a distal end of the cannula, such that it is located near an attachment point of the radiation spacer device to the cannula. The resistive coil may detach the radiation spacer device by thermally ablating the neck of the implantable balloon with the resistive coil.

In a seventeenth embodiment, the present disclosure relates to the radiation delivery system of any of the previous embodiments, wherein the fluid is a biodegradable hydrogel.

In an eighteenth embodiment, the present disclosure relates to the radiation delivery system of any of the previous embodiments, wherein the implantable balloon is a biodegradable polymer.

In a nineteenth embodiment, the present disclosure relates to the use of the radiation spacer device or the radiation delivery system of any of the previous embodiments in a patient.

In a twentieth embodiment, the present disclosure relates a method of protecting a non-targeted tissue from collateral radiation, the method comprising: inserting a radiation spacer device between a targeted tissue, wherein the targeted tissue is intended to receive radiation therapy, and the non-targeted tissue, the radiation spacer device comprising: an implantable balloon defining a cavity for holding a fluid therein, the implantable balloon having a flexible body and a neck, said neck defining an opening into the cavity, and a plurality of duckbill valves disposed within the opening into the cavity, wherein the plurality of duckbill valves are serially nested within one another and arranged relative to one another to restrict movement of the fluid out of the cavity; expanding the implantable balloon to create a separation between the targeted tissue and the non-targeted tissue, thereby protecting the non-targeted tissue from collateral radiation.

In a twenty first embodiment, the present disclosure relates to the method of any of the previous embodiments, wherein the targeted tissue is cancerous tissue and the non-targeted tissue is an adjacent organ.

In a twenty second embodiment, the present disclosure relates to the method of any of the previous embodiments, wherein the radiation spacer device is coupled to a fluid delivery apparatus.

In a twenty third embodiment, the present disclosure relates to the method of any of the previous embodiments, wherein the fluid delivery apparatus comprises a housing and a detachment mechanism.

In a twenty fourth embodiment, the present disclosure relates to the method of any of the previous embodiments, the method further comprising detaching the radiation spacer device from the fluid delivery apparatus using the detachment mechanism.

In a twenty fifth embodiment, the present disclosure relates to the method of any of the previous embodiments, wherein the detachment mechanism is a resistive coil.

In a twenty sixth embodiment, the present disclosure relates to the method of any of the previous embodiments, wherein detaching the radiation spacer device comprises thermally ablating the neck of the implantable balloon with the resistive coil.

In a twenty seventh embodiment, the present disclosure relates to the method of any of the previous embodiments, wherein expanding the implantable balloon comprises filling the cavity with the fluid.

In a twenty eighth embodiment, the present disclosure relates to the method of any of the previous embodiments, wherein the fluid is a biodegradable hydrogel.

In a twenty ninth embodiment, the present disclosure relates to the method of any of the previous embodiments, wherein filling the cavity with the fluid comprises passing the fluid from an injection assembly to the cavity.

In a thirtieth embodiment, the present disclosure relates to the method of any of the previous embodiments, wherein the injection assembly comprises an elongate member and a syringe, the syringe comprising a chamber for holding the fluid and a plunger.

In a thirty first embodiment, the present disclosure relates to the method of any of the previous embodiments, wherein passing the fluid from the injection assembly to the cavity comprises actuation of the plunger to dispense the fluid from the chamber, into the elongate member, and into the cavity.

In a thirty second embodiment, the present disclosure relates to use of the radiation spacer device and/or radiation delivery system of any of the previous embodiments to protect a non-targeted tissue from collateral radiation, the use comprising: inserting a radiation spacer device between a targeted tissue, wherein the targeted tissue is intended to receive radiation therapy, and the non-targeted tissue, the radiation spacer device comprising: an implantable balloon defining a cavity for holding a fluid therein, the implantable balloon having a flexible body and a neck, said neck defining an opening into the cavity, and a plurality of duckbill valves disposed within the opening into the cavity, wherein the plurality of duckbill valves are serially nested within one another and arranged relative to one another to restrict movement of the fluid out of the cavity; expanding the implantable balloon to create a separation between the targeted tissue and the non-targeted tissue, thereby protecting the non-targeted tissue from collateral radiation.

In a thirty third embodiment, the present disclosure relates to the use of any of the previous embodiments, wherein the targeted tissue is cancerous tissue and the non-targeted tissue is an adjacent organ.

In a thirty fourth embodiment, the present disclosure relates to the use of any of the previous embodiments, wherein the radiation spacer device is coupled to a fluid delivery apparatus.

In a thirty fifth embodiment, the present disclosure relates to the use of any of the previous embodiments, wherein the fluid delivery apparatus comprises a housing and a detachment mechanism.

In a thirty sixth embodiment, the present disclosure relates to the use of any of the previous embodiments, the use further comprising detaching the radiation spacer device from the fluid delivery apparatus using the detachment mechanism.

In a thirty seventh embodiment, the present disclosure relates to the use of any of the previous embodiments, wherein the detachment mechanism is a resistive coil.

In a thirty eighth embodiment, the present disclosure relates to the use of any of the previous embodiments, wherein detaching the radiation spacer device comprises thermally ablating the neck of the implantable balloon with the resistive coil.

In a thirty ninth embodiment, the present disclosure relates to the use of any of the previous embodiments, wherein expanding the implantable balloon comprises filling the cavity with the fluid.

In a fortieth embodiment, the present disclosure relates to the use of any of the previous embodiments, wherein the fluid is a biodegradable hydrogel.

In a forty first embodiment, the present disclosure relates to the use of any of the previous embodiments, wherein filling the cavity with the fluid comprises passing the fluid from an injection assembly to the cavity.

In a forty second embodiment, the present disclosure relates to the use of any of the previous embodiments, wherein the injection assembly comprises an elongate member and a syringe, the syringe comprising a chamber for holding the fluid and a plunger.

In a forty third embodiment, the present disclosure relates to the use of any of the previous embodiments, wherein passing the fluid from the injection assembly to the cavity comprises actuation of the plunger to dispense the fluid from the chamber, into the elongate member, and into the cavity.

Any embodiment is capable of being used in combination with, or separate from any other embodiment.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A radiation spacer device comprising:
an implantable balloon defining a cavity for holding a fluid therein, the implantable balloon having a flexible body and a neck, said neck defining an aperture into the cavity; and
a plurality of duckbill valves disposed within the aperture, wherein the plurality of duckbill valves are serially nested within one another and arranged relative to one another to restrict movement of the fluid out of the cavity.

2. The radiation spacer device of claim 1 wherein the implantable balloon is formed from a biodegradable polymer.

3. The radiation spacer device of claim 1 wherein the implantable balloon is expandable in response to an increasing amount of fluid introduced to the cavity.

4. The radiation spacer device of claim 1 wherein each duckbill valve of the plurality of duckbill valves comprises:
a base portion defining a first lumen portion; and
a bill portion extending distally from the base portion and defining a second lumen portion having a decreasing cross-sectional area from the first lumen portion to an outlet formed at a distal end of the duckbill valve;
wherein the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity.

5. The radiation spacer device of claim 4, wherein the bill portion of the second duckbill valve is angularly rotated relative to the bill portion of the first duckbill valve by 90 degrees.

6. The radiation spacer device of claim 1 wherein the fluid comprises a biodegradable hydrogel.

7. The radiation spacer device of claim 1, wherein the radiation spacer device has a bioadhesive coating or physical mechanism for decreasing the mobility of the radiation spacer within an insertion site disposed on an exterior surface of the flexible body.

8. A radiation spacer delivery system, comprising:
a radiation spacer device comprising:
an implantable balloon defining a cavity for holding a fluid therein, the implantable balloon having a flexible body and a neck, said neck defining an opening into the cavity, and
a plurality of duckbill valves disposed within the opening into the cavity, wherein the plurality of duckbill valves are serially nested within one another and arranged relative to one another to restrict movement of the fluid out of the cavity;
an injection assembly comprising:
an elongate member, and
a syringe comprising a chamber and a plunger, wherein the chamber is fluidically coupled to the elongate member; and
a fluid delivery apparatus comprising:
a housing, comprising a cannula, a support member, and an actuator, and a detachment mechanism.

9. The radiation spacer delivery system of claim 8, wherein each duckbill valve of the plurality of duckbill valves comprises:
a base portion defining a first lumen portion; and
a bill portion extending distally from the base portion and defining a second lumen portion having a decreasing cross-sectional area from the first lumen portion to an outlet formed at a distal end of the duckbill valve;
wherein the bill portion of a first duckbill valve extends into and is retained in the first lumen portion of a second duckbill valve, such that the bill portion of the first duckbill valve is angularly rotated relative to the bill portion of the second duckbill valve, thereby preventing leakage through the plurality of duckbill valves to restrict the movement of the fluid out of the cavity.

10. The radiation spacer delivery system of claim 9, wherein the bill portion of the second duckbill valve is angularly rotated relative to the bill portion of the first duckbill valve by 90 degrees.

11. The radiation spacer delivery system of claim 8, wherein the detachment mechanism comprises a resistive coil.

12. The radiation spacer delivery system of claim 8, wherein the fluid is a biodegradable hydrogel.

13. The radiation spacer delivery system of claim 8, wherein the implantable balloon is a biodegradable polymer.

14. A method of protecting a non-targeted tissue from collateral radiation, the method comprising:
inserting a radiation spacer device between a targeted tissue, wherein the targeted tissue is intended to receive radiation therapy, and the non-targeted tissue, the radiation spacer device comprising:
an implantable balloon defining a cavity for holding a fluid therein, the implantable balloon having a flexible body and a neck, said neck defining an opening into the cavity, and
a plurality of duckbill valves disposed within the opening into the cavity, wherein the plurality of duckbill valves are serially nested within one another and arranged relative to one another to restrict movement of the fluid out of the cavity;
expanding the implantable balloon to create a separation between the targeted tissue and the non-targeted tissue, thereby protecting the non-targeted tissue from collateral radiation.

15. The method of claim 14, wherein the targeted tissue is cancerous tissue and the non-targeted tissue is an adjacent organ.

16. The method of claim 14, wherein the radiation spacer device is coupled to a fluid delivery apparatus.

17. The method of claim 16, wherein the fluid delivery apparatus comprises a housing and a detachment mechanism.

18. The method of claim 17, further comprising detaching the radiation spacer device from the fluid delivery apparatus using the detachment mechanism.

19. The method of claim 18, wherein the detachment mechanism is a resistive coil.

20. The method of claim 19, wherein detaching the radiation spacer device comprises thermally ablating the neck of the implantable balloon with the resistive coil.

21. The method of claim 14, wherein expanding the implantable balloon comprises filling the cavity with the fluid.

22. The method of claim 21, wherein the fluid is a biodegradable hydrogel.

23. The method of claim 22, wherein filling the cavity with the fluid comprises passing the fluid from an injection assembly to the cavity.

24. The method of claim 23, wherein the injection assembly comprises an elongate member and a syringe, the syringe comprising a chamber for holding the fluid and a plunger.

25. The method of claim 24, wherein passing the fluid from the injection assembly to the cavity comprises actuation of the plunger to dispense the fluid from the chamber, into the elongate member, and into the cavity.
